(19) United States
(12) Patent Application Publication
(10) Pub. No.: US 2024/0377354 A1
(43) Pub. Date: Nov. 14, 2024

(54) CIRCUITRY FOR SENSOR MEASUREMENT

(71) Applicant: Cirrus Logic International Semiconductor Ltd., Edinburgh (GB)

(72) Inventors: John P. LESSO, Edinburgh (GB); Toru IDO, Kanagawa (JP)

(73) Assignee: Cirrus Logic International Semiconductor Ltd., Edinburgh (GB)

(21) Appl. No.: 18/611,311

(22) Filed: Mar. 20, 2024

Related U.S. Application Data
(60) Provisional application No. 63/500,983, filed on May 9, 2023.

(51) Int. Cl. G01N 27/416; G01N 27/403

(52) U.S. Cl. CPC G01N 27/4163; G01N 27/403

(57) ABSTRACT

Circuitry for measuring a characteristic of a sensor having a reactive impedance, the circuitry comprising: a first integrator having a first integrator input, a first integrator output, and an integrating element coupled between the first integrator input and the first integrator output, the integrating element comprising the sensor; And processing circuitry configured to: determine a state of the sensor based on an output signal derived from the first integrator output.

### TECHNICAL FIELD

The present disclosure relates to circuitry for measuring characteristics in sensors having a reactive impedance.

### BACKGROUND

Electrochemical sensors are widely used for the detection of one or more particular chemical species, analytes, as an oxidation or reduction current. Such sensors comprise an electrochemical cell, consisting of two or more electrodes configured for contact with an analyte whose concentration is to be ascertained. Such sensors also comprise circuitry for driving one or more of the electrodes and for measuring a response at one or more of the electrodes.

Conventional drive and measurement circuitry in electrochemical sensors comprises several amplifiers, feedback and/or feedback loops in addition to other processing circuitry, such as analog-to-digital converters (ADCs). Such circuitry can take up a large amount of space on-chip, as well as being relatively process intensive, thereby utilising large amounts of power. When electrochemical sensors are battery powered, for example when used in continuous glucose monitoring, it is desirable for such sensors to be as small as possible and use as little power as possible.

### SUMMARY

Embodiments of the disclosure aim to address or at least ameliorate one or more of the above issues by reducing the overall power and size of the drive and measurement circuitry required to measure analyte concentration in electrochemical sensors. Specifically, embodiments of the disclosure utilise the filtering characteristics of the electrochemical cell (or any other sensor comprising a reactive impedance) as the filter in an analog-to-digital converter (ADC) circuit, thus saving both power and space. By reducing the size and power of drive and measurement circuitry, multiple electrochemical sensors can be integrated into a single device, thereby either providing redundancy or enabling the sensing of multiple analytes in a single chip. Multiple analyte sensors may be particularly advantageous in applications such as continuous glucose monitoring, where it may be desirable to measure concentrations of glucose, ketones, oxygen, lactate, and the like. Embodiments of the present disclosure may be applicable to other types of sensors, such as gas sensors and aptamer based sensors by way of non-limiting example only. Since such sensors present a largely capacitive load, embodiments of the present disclosure exploit that capacitive nature by using the electrochemical cell as an integrating capacitor in a feedforward path of an ADC.

According to a first aspect of the disclosure, there is provided circuitry for measuring a characteristic of a sensor having a reactive impedance, the circuitry comprising: a first integrator having a first integrator input, a first integrator output, and an integrating element coupled between the first integrator input and the first integrator output, the integrating element comprising the sensor; and processing circuitry configured to determine a state of the sensor based on an output signal derived from the first integrator output.

The state of the sensor may comprise a characteristic of the sensor, a fault at the sensor, or a condition of an analyte present at the sensor.

The processing circuitry may be configured to low-pass filter the output signal to determine a first aspect of the state of the sensor.

The processing circuitry may be configured to high-pass filter the output signal to determine a second aspect of the state of the sensor.

The processing circuitry may be configured to band-pass filter the output signal to determine a third aspect of the state of the sensor at one or more band-pass frequencies defined by the band-pass filtering.

The processing circuitry may be configured to apply a Fourier transform to the output signal to determine the state of the sensor as a function of frequency.

During derivation of the output signal, the processing circuitry may be configured to hold first and second electrodes of the sensor at first and second predetermined voltages.

The circuitry of any one of the preceding claims, wherein the sensor is a capacitive sensor.

The circuitry of any one of the preceding claims, wherein the sensor is an inductive sensor.

The circuitry may comprise an analog-to-digital converter (ADC), comprising, the ADC comprising an ADC output configured to output the output signal; and the first integrator. In other words, the first integrator may form part of the ADC comprised in the circuitry.

The ADC may be a sigma-delta ADC. Alternatively, the DAC may be a pulse width modulated (PWM) ADC.

A digital-to-analog converter (DAC) may be provided in a feedback path between the ADC output and the first integrator input.

The processing circuitry may be configured to: derive a noise transfer function of the ADC from the output signal. The state of the sensor may then be derived based on the noise transfer function.

The ADC may comprise a quantizer comprising a quantiser input coupled to the first integrator output and a quantiser output coupled to the ADC output. The quantiser may be configured to derive the output signal based on the first integrator input. The quantizer may comprise a comparator, such as a hysteretic comparator. The circuitry may further comprise control circuitry configured to adapt a hysteresis of the comparator based on a limit cycle frequency or limit cycle period of the first quantizer output.

The quantizer may be a single-bit quantiser or a multi-bit quantiser. Where the quantiser is a multi-bit quantiser, the DAC may comprise a finite impulse response (FIR) DAC.

The circuitry may further comprise dither circuitry configured to add noise at one or more of: the first integrator input; the ADC output; the first integrator output; and the feedback path between the ADC input and the ADC output.

The circuitry may further comprise second integrator having a second integrator input coupled to the first integrator output and a second integrator output coupled to the quantiser input.

In some embodiments, the sensor may comprise an electrochemical cell. The electrochemical cell may comprise an analyte measurement cell (e.g., an electrochemical sensor or a gas sensor). Alternatively, the electrochemical cell may comprise a battery cell. The state of the sensor may comprise an impedance of the electrochemical cell, such as a reactive impedance.

The electrochemical cell may comprise a potentiostat. The potentiostat may comprise at least one working electrode and at least one counter electrode. The at least one counter electrode may be coupled to the first integrator input and the at least one working electrode may be coupled to the first integrator output. The potentiostat may comprise a reference electrode. The reference electrode may be coupled to the first integrator input and the at least one working electrode may be coupled to the first integrator output.

The electrochemical cell may be configured to sense a plurality of different analytes. For example, the plurality of different analytes may be selected from a list comprising two or more of glucose, lactates and ketones.

The electrochemical cell may comprise a battery comprising an anode coupled to the first integrator input and a cathode coupled to the first integrator output.

According to another aspect of the disclosure, there is provided circuitry for measuring a characteristic of a sensor having a reactive impedance (e.g. an electrochemical cell), the circuitry comprising: a first integrator having a first integrator input, a first integrator output, and an integrating element coupled between the first integrator input and the first integrator output, the integrating element comprising the sensor; and processing circuitry configured to decode an output signal derived from the first integrator output to determine a state of the sensor.

According to another aspect of the disclosure, there is provided circuitry comprising: a sensor having a reactive impedance; the sensor arranged as an integrating element of the circuit (e.g. as an integrating capacitive element of the circuit); wherein an integrated output of the integrating element is decoded to determine a state of the sensor.

According to another aspect of the disclosure, there is provided an electronic device, comprising any of the circuitry described above.

The electronic device may comprise a continuous glucose monitor. Additionally or alternatively, the electronic device may comprise one of a wearable device, a medical device, an augmented reality headset, a virtual reality headset, a mobile computing device, a laptop computer, a tablet computer, a games console, a remote control device, a home automation controller, a domestic appliance, a toy, a robot, an audio player, a video player, a mobile telephone, and a smartphone.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the present disclosure will now be described by way of non-limiting examples with reference to the drawings, in which:

FIG. 1 illustrates a schematic diagram and electrical equivalent circuit for a three-electrode electrochemical cell;

FIG. 2 illustrates a schematic diagram and electrical equivalent circuit for a three-electrode electrochemical cell;

FIG. 3 illustrates a schematic diagram and electrical equivalent circuit for a two-electrode electrochemical cell;

FIG. 4 is a schematic diagram of an example prior art measurement circuit;

FIG. 5 is a schematic diagram of an example prior art sigma-delta ADC;

FIG. 6 is a schematic diagram of example measurement circuitry for measuring a characteristic of a two-electrode electrochemical cell;

FIG. 7 is a schematic diagram of the measurement circuitry shown in FIG. 6, with the electrochemical cell replaced with its equivalent circuit;

FIG. 8 is a schematic diagram of example measurement circuitry for measuring a characteristic of a three-electrode electrochemical cell;

FIG. 9 is a schematic diagram of the measurement circuitry shown in FIG. 8, with the electrochemical cell replaced with its equivalent circuit;

FIG. 10 illustrates a variation of measurement circuitry of FIG. 6 comprising circuitry for introducing dither;

FIG. 11 illustrates a variation of drive and measurement circuitry of FIG. 6 comprising circuitry for introducing dither;

FIG. 12 illustrates a variation of measurement circuitry of FIG. 6 comprising circuitry for introducing dither;

FIG. 13 illustrates a variation of measurement circuitry of FIG. 6 comprising circuitry for introducing dither with pulse width modulation (PWM);

FIG. 14 illustrates a variation of measurement circuitry of FIG. 6 comprising circuitry for introducing dither and hysteresis;

FIG. 15 illustrates a variation of measurement circuitry of FIG. 6 comprising circuitry for decoding an output;

FIG. 16 illustrates a variation of measurement circuitry of FIG. 6 the digital-to-analog converter (DAC) replaced with a finite impulse response (FIR) DAC;

FIG. 17 is a schematic diagram of example measurement circuitry comprising a second order integrating loop; and

FIG. 18 is a schematic diagram of example measurement circuitry comprising a second order integrating loop and an additional control loop.

### DESCRIPTION OF EMBODIMENTS

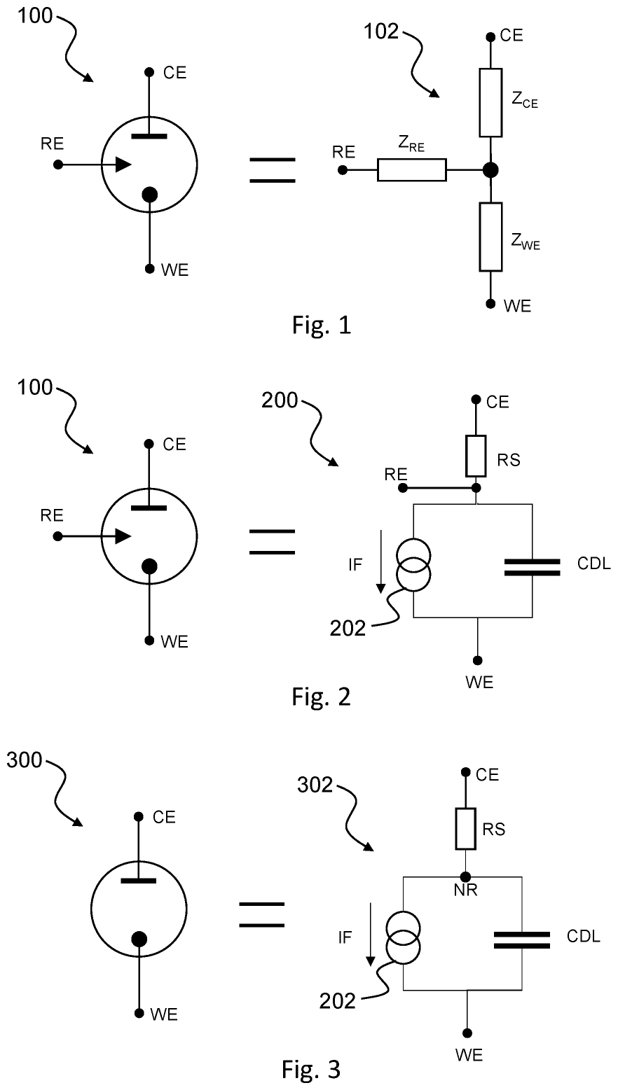

FIG. 1 is a schematic diagram of an electrochemical cell 100 comprising three electrodes, namely a counter electrode CE, a working electrode WE and a reference electrode RE. FIG. 1 also shows an equivalent circuit 102 for the electrochemical cell comprising a counter electrode inductance ZCE, a working electrode inductance ZWE and a reference electrode inductance ZRE.

To determine a characteristic of the electrochemical cell, and therefore an analyte concentration, a measurement current is injected at the counter electrode CE and a current at the working electrode is measured. The reference electrode RE is used to measure a voltage drop between the working electrode WE and the reference electrode RE. The measurement current is then adjusted to keep voltage drop constant. As the resistance in the cell 100 increases, the voltage drop measured at the reference electrode increases. In response, the measurement current injected at the counter electrode CE is decreased. Likewise, as the resistance in the cell 100 decreases, the voltage drop measured at the reference electrode decreases. In response, the measurement current injected at the counter electrode CE is increased. Thus the electrochemical cell 100 reaches a state of equilibrium where the voltage drop between the reference electrode RE and the working electrode WE is maintained constant. Since the current injected at the counter electrode CE and the voltage drop are known, the resistance of the cell 100 can be ascertained.

FIG. 2 illustrates an alternative equivalent circuit 200 for the three-electrode electrochemical cell 100. The circuit 200 comprises a resistance RS between the counter electrode CE and the reference electrode RE, and a current source 202 and a double layer capacitance CDL provided parallel between the reference electrode RE and the working electrode WE. During operation, the current source 202 drives a current IF to the working electrode WE.

FIG. 3 illustrates a two-electrode electrochemical cell 300 which is a variation of the cell 100 with the omission of the reference electrode RE. An equivalent circuit 302 for the two-electrode cell 300 is also shown. The equivalent circuit 302 of FIG. 3 differs from the equivalent circuit 202 of FIG. 2 in that the resistance RS is coupled to a reference node NR and the double layer capacitance CDL and the current source 202 are each coupled between the reference node NR and the working electrode WE.

In the equivalent circuits shown in FIG. 3, the double layer capacitance CDL dominates the characteristic of each of the electrochemical cells 100, 300. Embodiments of the present disclosure aim to exploit this capacitive nature of the cells 100, 300 by building an ADC around an electrochemical cells 100, 300, using the double layer capacitance CDL as an integrating element. Operation of example embodiments will be described in more detail below.

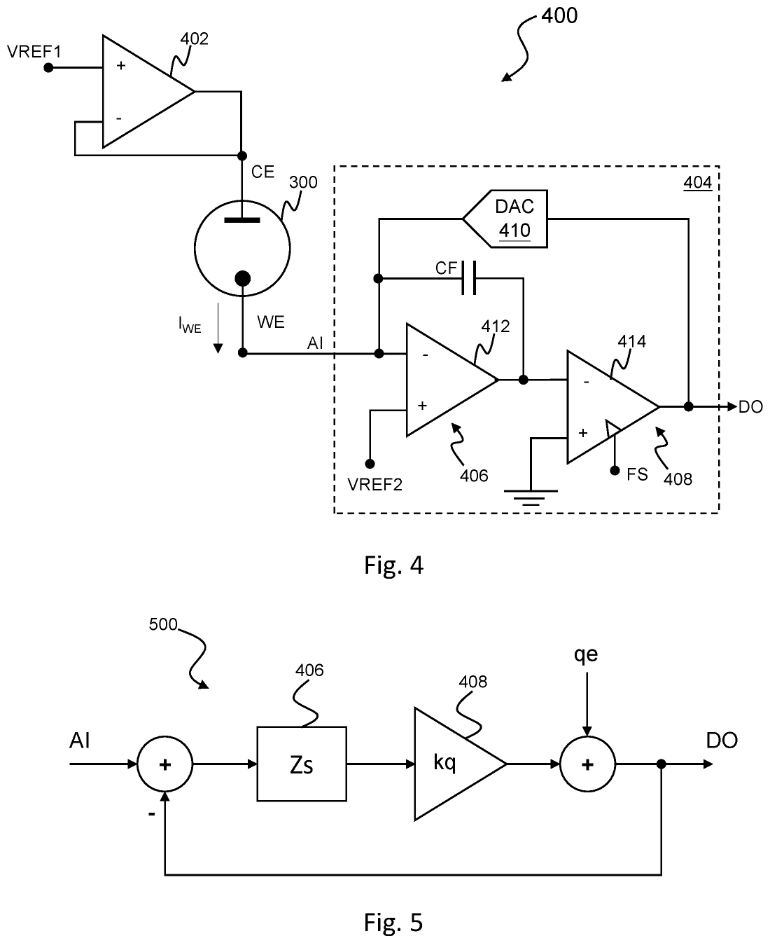

FIG. 4 illustrates an example drive and measurement circuit 400 which is configured to characterise the two-electrode electrochemical cell 300, specifically for measuring an analyte concentration in the electrochemical cell 300 shown in FIG. 3. The circuit 400 comprises a comparator 202 and an ADC 410. A non-inverting input of the comparator 402 is coupled to a first reference voltage VREF1. An inverting input of the comparator 402 is coupled to the counter electrode CE of the cell 300. An output of the comparator 402 is also coupled to the counter electrode CE. Thus, the comparator 402 is configured to inject the measurement current into the cell 300 which is proportional to the difference between the first reference voltage VREF1 and the voltage at the counter electrode CE. As such, the comparator acts to maintain the voltage between the counter electrode CE and the working electrode WE close to the first reference voltage VREF1.

The ADC 404 is configured to generate a digital output DOUT representative of a working electrode current IWE measured at the working electrode WE. Whilst, in the embodiment shown, the ADC 404 is a sigma-delta ADC, embodiments of the present disclosure are not limited to such ADC architectures and may be equally applicable to other ADC architectures, such as a pulse width modulation (PWM) ADC.

The ADC 404 comprises an integrator 406, a quantiser 408 and a DAC 410. The integrator 406 comprises an op-amp 412 comprising an inverting input coupled to the working electrode WE, a non-inverting input coupled to a second reference voltage VREF2, and an output coupled to an inverting input of the quantiser 408. The integrator 406 further comprises a feedforward capacitor CF coupled between the inverting input and the output of the op-amp 412. In this example, the quantiser 408 comprises a comparator 414 clocked at a sampling frequency FS. As noted above, the inverting input of the comparator 414 (and the quantiser 408) is coupled to the output of the integrator 406. A non-inverting input of the comparator 414 is coupled to a reference voltage (in this case ground GND), and an output. The DAC 410 is coupled between the output of the quantiser 408 and the inverting input of the op-amp 412 of the integrator 406.

FIG. 5 is a system diagram 500 equivalent to the ADC 404 shown in FIG. 4, comprising the integrator 406 having an impedance Zs and the quantiser 408 having a quantisation gain kq. The DAC 410 is represented in FIG. 4 as the negative feedback look between the output of the ADC 404 and the input of the integrator 406. Quantisation error qe, due to sampling by the quantiser 408, is added to the output signal DO.

The system transfer function (STF) for the ADC 404 assuming zero quantisation error qe is given by the following equation where AI is the analog input of the ADC 404 and DO is the digital output of the ADC 404.

$$STF = \frac{AI}{DO}$$

The noise transfer function (NTF) with the analog input signal AI not present (or equal to zero) is given by the following equation.

$$NTF = \frac{DO}{{q}_{e}}$$

Which can be re-written in terms of the impedance Zs and the quantisation gain kq as follows.

$$NTF = \frac{1}{1 + {Z}_{s} {k}_{q}}$$

Hence, if the quantisation gain kq and the NTF are known, it is possible to determine the impedance Zs of the integrator 406.

As such, referring again to FIG. 4, by replacing the feedforward resistor CF of the integrator 406 with an electrochemical cell, such as the cells 100, 300 described above, if the impedance of the op-amp 412 is known, the impedance of the feedforward capacitance (i.e., the cell) can be ascertained.

For the ADC 404 to operate accurately, several requirements should be met in the design of the ADC 404. Firstly, as mentioned above, the ADC 404 should operate with a known quantisation gain kq. This can be achieved, for example, through used of dither or through the use of a PWM quantiser (both techniques described in more detail below). Secondly, the statistics of the quantisation error qe should be known or derivable. For example, when the ADC 404 is controlled to have a known quantisation gain kq (such as with dither applied) the quantisation error qe will have a white spectrum (i.e., flat spectrum) with a power of approximately Δ²/12 where Δ is the quantisation step of the quantiser 408 (i.e., quantiser). Where a PWM quantiser is implemented as the quantiser 408 (i.e., in place of the clocked comparator 414) the quantisation error qe will be given by the power at the limit cycle and harmonics of the PWM quantiser.

In view of the above, with the analog input signal AI absent, we can assume that the power spectrum of the output y is a proxy for the NTF with a quantiser dependent scaling for qe. Hence, if the NTF is measured (via the output y) and the quantisation gain kq is known, then we can estimate the impedance Zs as follows:

$${Z}_{s}^{*} = \frac{1 - NTF}{{k}_{q} NTF} = \frac{{q}_{e} - y}{{k}_{q} y}$$

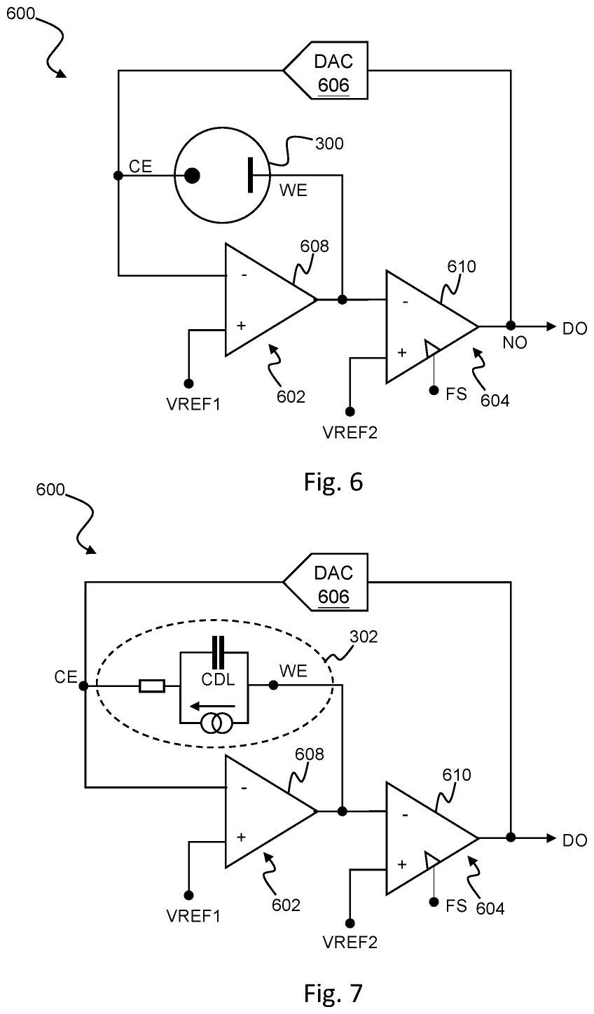

Embodiments of the present disclosure aim to at least partially replace the impedance Zs of the integrator 406 with an electrochemical cell, such as the cells 100, 300 shown in FIGS. 1 and 3. Various example implementations of such circuitry for characterising electrochemical cells will now be described with reference to FIGS. 6 to 9.

FIG. 6 is a schematic diagram of a measurement circuit 600 comprising the electrochemical cell 300 of FIG. 3. The measurement circuit 600 comprises an integrator 602, a quantiser 604 and a DAC 606.

The integrator 602 comprises an op-amp 608 having a non-inverting input, an inverting input and an output. The non-inverting input of the op-amp 608 is coupled to a first reference voltage VREF1. The electrochemical cell 300 is provided in a feedforward path between the non-inverting input and the output of the op-amp 608. For example, the working electrode WE of the cell 300 is coupled to output of the op-amp 608 and the counter electrode CE is coupled to the non-inverting input of the op-amp 608.

In this example, the quantiser 604 comprises a clocked comparator 610 having a non-inverting input coupled to the output of the integrator 602, an inverting input coupled to a second reference voltage VREF2 (which in some examples may be at ground GND), and an output coupled to an output node NO of the measurement circuit 600.

The DAC 606 comprise an input coupled between the output node NO and an output coupled to the non-inverting input of the op-amp 608 (and therefore also coupled to the counter electrode CE of the cell 300.

FIG. 7 is a schematic diagram of the measurement circuit 600 with the cell 300 replaced with its equivalent circuit 302. It can be seen that the double layer capacitor CDL acts as an integrating capacitor in the feedforward loop of the integrator 602. As discussed above, since the impedance Zs of the integrator can be ascertained from the digital output DO and the impedance of the op-amp 608 is known, the double layer capacitance CDL of the cell 300 can be ascertained from the digital output DO.

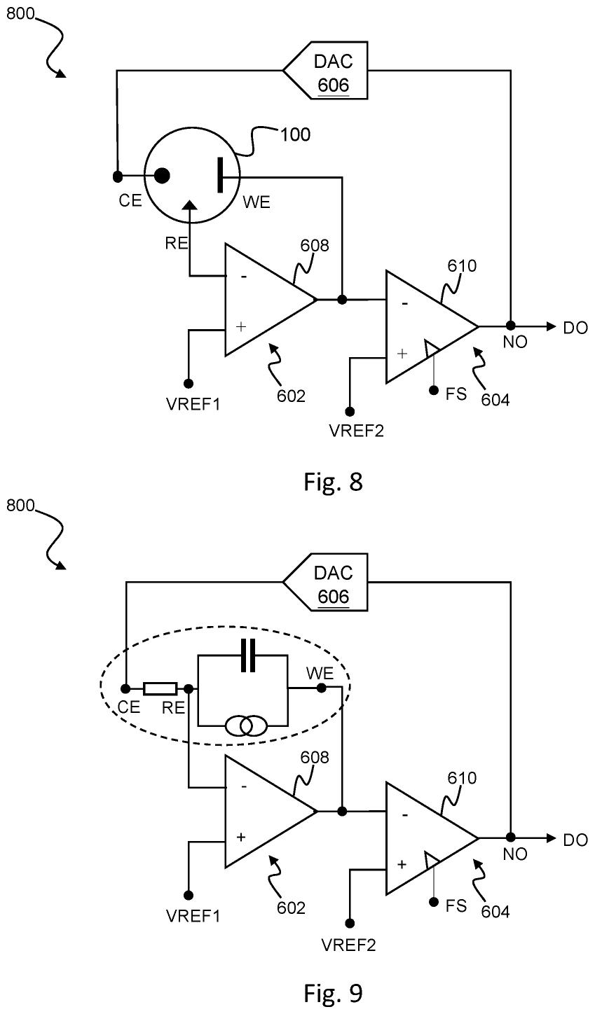

FIG. 8 is a is a schematic diagram of a measurement circuit 800 which is a variation of the measurement circuit 600 of FIG. 6 comprising the three-electrode electrochemical cell 100 of FIG. 1, in place of the two-electrode cell 300 of FIG. 3. Like parts have been denoted with like reference numerals. The measurement circuit 800 of FIG. 8 differs from that of FIG. 6 in that the output of the DAC 606 is not the inverting input of the op-amp 608. Instead, the reference electrode RE of the electrochemical cell 100 is coupled to the inverting input of the op-amp 608.

In the arrangement shown in FIG. 8, the reference electrode RE of the cell 100 is used to ensure a suitable voltage is applied across the cell 100 taking into account the impedance between the counter electrode CE and the reference electrode RE of the cell 100. The op-amp 608 operates as a transconductance element (or gM cell) producing an output current proportional to a difference between a voltage at the reference electrode RE and the first reference voltage VREF1.

FIG. 9 is a schematic diagram of the measurement circuit 800 with the cell 100 replaced with its equivalent circuit 202. It can be seen that the double layer capacitor CDL acts as an integrating capacitor in the feedforward loop of the integrator 602. As discussed above, since the impedance Zs of the integrator can be ascertained from the digital output DO and the impedance of the op-amp 608 is known, the double layer capacitance CDL of the cell 100 can be ascertained from the digital output DO.

As noted above, to aid in accurately determining the impedance Zs of the integrator 602 and therefore characteristic(s) of the various cells 100, 300 described herein, the various measurement circuits 600, 800 preferably include additional circuitry for stabilizing quantisation gain kq and quantisation error qe so that they are each derivable.

In some embodiments, stabilisation of quantisation gain kq and error qe may be achieved using dither. As is known in the art, dither is an intentionally applied form of noise used to randomize quantisation error. Various example implementations of dither are described below which are based on the measurement circuit 600 of FIG. 6 comprising the two-electrode cell 300. It will be appreciated that these implementations are equally applicable to the measurement circuit 800 of FIG. 8 comprising the three-electrode cell 100.

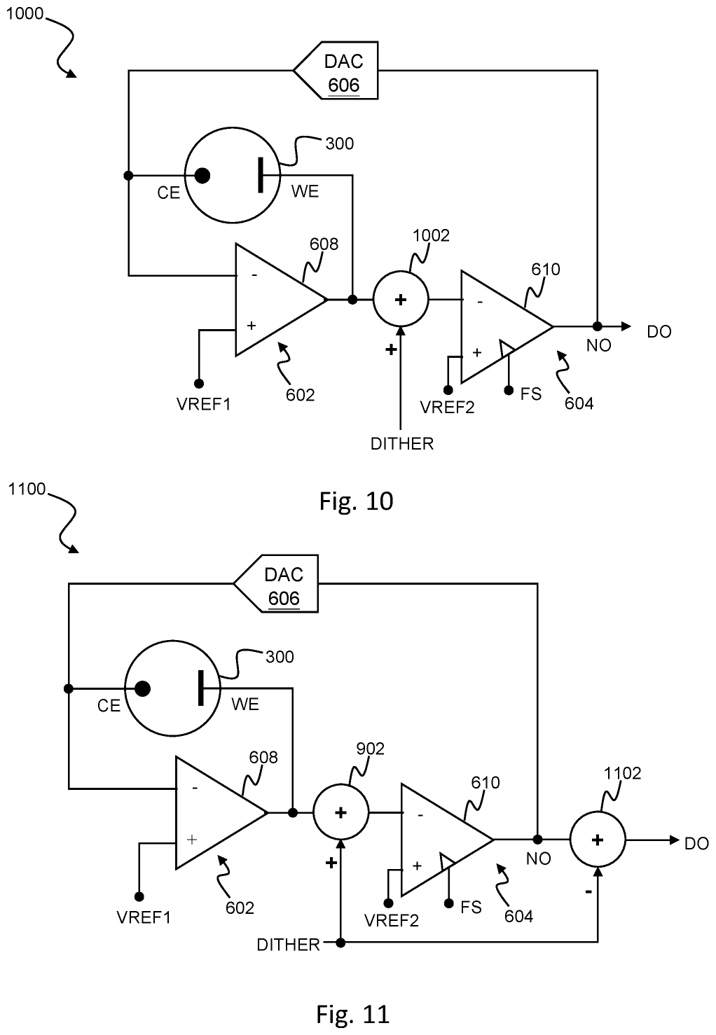

FIG. 10 is a schematic diagram of a measurement circuit 1000 which is a variation of the circuit 600 of FIG. 6, like parts denoted with like numerals, implementing non-subtractive dither. The measurement circuit 1000 comprises an adder 1002 between the output of the integrator 602 and the non-inverting input of the quantiser 604. Dither (e.g., randomised noise) is added by the adder 1002 to the integrated signal output from the integrator 602.

FIG. 11 is a schematic diagram of a measurement circuit 1100 which is a variation of the circuit 1000 of FIG. 1 implementing subtractive dither. A subtraction module 1102 is provide at the output node NO. The dither added at the adder 902 is subtracted from the signal output at the output node NO to form the digital output DO. Thus, the noise added by the adder 1002 to randomize the quantisation error qe is removed from the digital output DO.

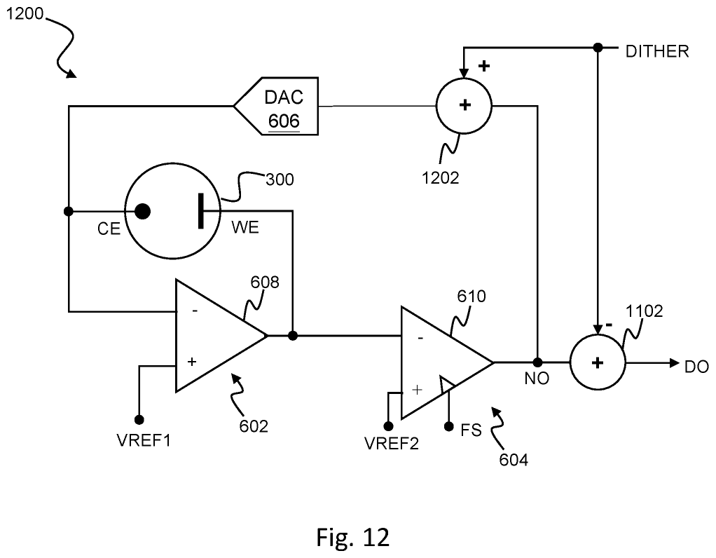

FIG. 12 is a schematic diagram of a measurement circuit 1200 which is a variation of the measurement circuit 1100 of FIG. 11, like parts denoted with like numerals. In the measurement circuit 1200, instead of providing the adder 902 at the output of the integrator 602, an adder 1202 is provided between the output node NO and the input of the DAC 606. Dither is thus injected into the feedback path comprising the DAC 606 and subtracted from the output by the subtraction module 1102. An advantage of this arrangement is that the addition and removal of dither is performed in the digital domain and can be more easily implemented and adjusted when compared to the addition/removal of dither in the analog domain.

In a variation of the circuit 1200 shown in FIG. 12, dither may additionally or alternatively be added at the output of the DAC 606.

As mentioned above with reference to FIG. 5, the quantiser 408 may be implemented as a PWM quantiser. In such embodiments, dither may be applied to the triangular wave used to generate the PWM output from the quantiser 408.

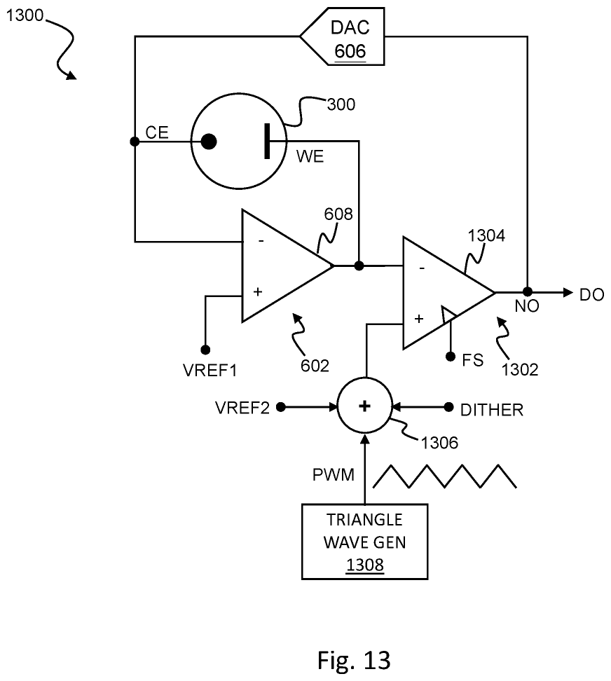

FIG. 13 is a schematic diagram of a measurement circuit 1300 which is a variation of the circuit 600 of FIG. 6 in which the quantiser 408 has been replaced with a quantiser 1302 which implements PWM quantisation. Like parts are denoted with like numerals. Like the quantiser 408 of FIG. 6, the quantiser 1302 is coupled between the output of the op-amp 608 and the output node NO. The quantiser 1302 comprises a comparator 1304, an adder 1306 and a triangle wave generator 1308. The comparator 1304 has an inverting input coupled to the output of the op-amp 608, an output coupled to the output node NO and a non-inverting input coupled to the output of the adder 1306. The triangle wave generator 1308 is configured to generate a triangle wave which is provided as an input to the adder 1306. The adder 1306 also receives as inputs the second reference voltage VREF2 in addition to dither. In providing a triangle wave to the non-inverting input of the comparator 1304, the comparator outputs a square wave with a fixed frequency but having a duty cycle which is proportional to the signal at the inverting input of the comparator 1304. Dither is then introduced to this output square wave via the non-inverting input of the comparator 1304.

In a variation of the circuit 1300 shown in FIG. 13, a dither signal may be provided to the triangle wave generator 1308 which may then be configured to generate a dither triangle wave to be provided to the comparator 1304 (instead of subsequently adding dither via the adder 1306 to the triangle wave generated without dither).

It will be appreciated that the circuit 1300 may be implemented without dither, implementing fixed carrier PWM quantisation based on the triangle wave generated by the triangle wave generator 1308.

In some embodiments, circuit 1300 may be switchable between a lower power mode in which the quantiser 1302 is implemented as a single bit quantiser and in a higher power mode in which the quantiser 1302 is operated with fixed carrier (e.g., using the triangle wave).

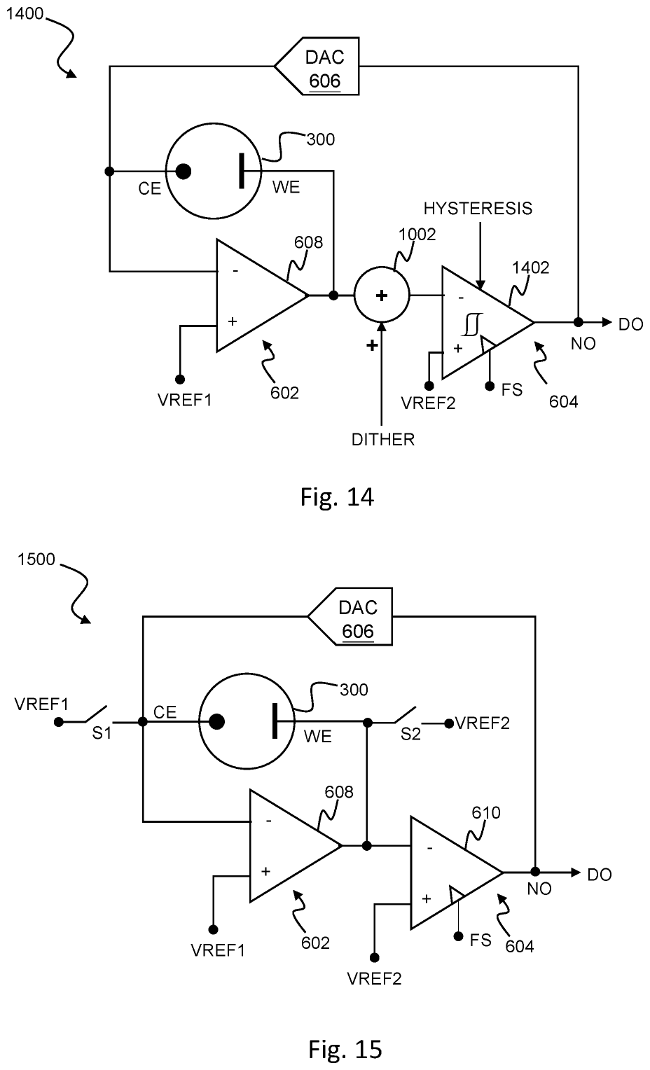

FIG. 14 is a schematic diagram of a measurement circuit 1400 which is a variation of the circuit 1000 of FIG. 10, like parts being denoted like numerals. When compared to the circuit 1000 of FIG. 10, in the measurement circuit 1400 the comparator 610 has been replaced with a hysteretic comparator 1402. In the embodiment shown, dither may be added by the adder 1002 provided prior to the comparator 1402 in the signal chain. Additionally or alternatively to adding dither at the adder 1002, dither may be added via modulation of hysteresis of the comparator (either by delay or by changes in voltage). In some embodiments, the adder 1002 may be omitted and any dither may be applied only by adjusting parameters of the comparator 1402.

It will be appreciated that to sample and decode the digital output DO, it may be necessary to hold the counter and working electrode CE, WE at the first and second reference voltage VREF1, VREF2 respectively.

FIG. 15 schematically illustrates a measurement circuit 1500 which is a variation of the measurement circuit 600 of FIG. 6, like parts denoted like reference numerals. First and second hold switch S1, S2 are provided. The first hold switch S1 is coupled between the counter electrode and the first reference voltage VREF1. The second hold switch S2 is coupled between the working electrode and the second reference voltage VREF2. During samples of the digital output DO, the first and second hold switches S1, S2 may be closed to maintain voltage difference between the output and the inverting input of the op-amp 608 constant. With the switches S1, D2 closed in this hold mode, the op-amp 608 and the comparator 604 may be switched off, thereby reducing overall power consumption of the measurement circuit 1500. It will be appreciated that the first and second hold switches S1, S2 may be implemented in any of the circuits described herein, the switches S1, S2 configured to hold the counter electrode CE and working electrode WE of respective cells 100, 300 at predefined voltage levels.

The circuits 600, 700, 800, 900, 1000, 1100, 1200, 1300, 1400, 1500 described above all implement a single bit DAC 606. To improve robustness to clock jitter, embodiments of the present disclosure may implement multibit behaviour, for example with the addition of a finite impulse response (FIR) filter.

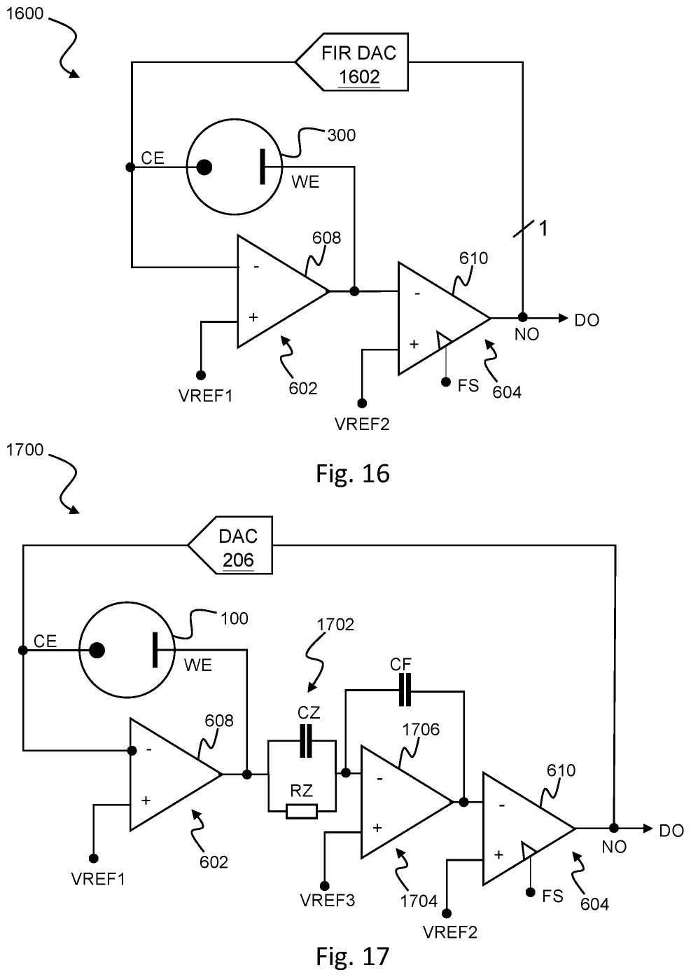

FIG. 16 is a schematic diagram of a measurement circuit 1600 which is a variation of the circuit 600 of the FIG. 6 in which the DAC 606 has been replaced with a FIR DAC 1602. The FIR DAC 1602 may be implemented as separate FIR filter and DAC elements or as a single element, performing digital to analog conversion simultaneously to FIR filtering. The single bit output at the output node NO is provided to the FIR DAC 1602 which sums delayed and scaled versions of the output on that single bit output to create a multibit digital signal which is the converted to an analog signal representing the digital output signal DO and provided to the inverting input of the op-amp 608. In a further variation of this arrangement, the quantiser 604 may be replaced with a multi-bit quantizer configured to output a multi-bit digital output which may be used by the FIR DAC 1602 to generate an analog representation of the multi-bit digital output. Multi-bit quantisers are known in the art and so will not be described in more detail here.

The circuits 600, 700, 800, 900, 1000, 1100, 1200, 1300, 1400, 1500, 1600 described above all implement a first order ADC loop. In some embodiments, it may be advantageous to implement higher order integration loops.

FIG. 17 is a schematic diagram of a measurement circuit 1700 which is a variation of the circuit 600 of FIG. 6, like parts being denoted like reference numerals. The circuit 1700 differs from the circuit 600 of FIG. 6 with the addition of a filter 1702 and a second integrator 1704 provided between the output of the integrator 204 and the inverting input of the quantiser 604.

The filter 1702 comprises a zero resistor RZ and a zero capacitor CZ arranged in parallel between the output of the integrator 602 and the inverting input of the second integrator 1704.

The integrator 1704 comprises a second op-amp 1706 having the inverting input (which is coupled to the filter 1702), a non-inverting input coupled to a third reference voltage VREF3, and an output coupled to the inverting input of the quantiser 604.

The filter 1702 is provided to stabilise the loop by adding a zero to the transfer function between the counter electrode CE and the output node NO.

It will be appreciated that in the arrangement in FIG. 17, direct control of the working electrode WE can no longer be achieved. To compensate for loss of control of the working electrode, one or more approaches can be taken. For example, the working electrode voltage may be modulated by a function of a state of the second integrator 1704 (e.g., Rz or DO). Alternatively, an additional loop may be added to adjust the working electrode voltage VWE at the working electrode WE as a function of the measured voltage at the working electrode WE.

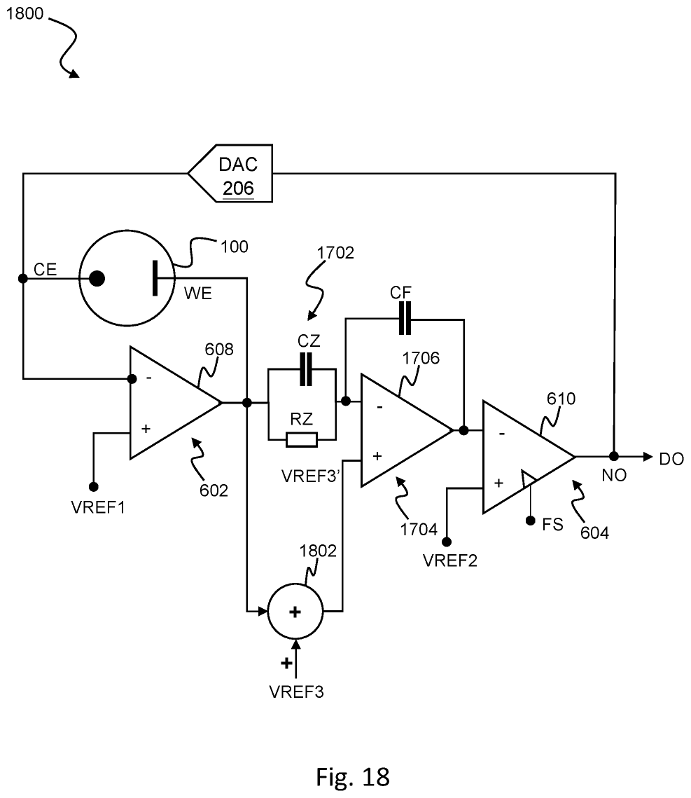

FIG. 18 is a schematic diagram of a measurement circuit 1800 which is a variation of the circuit 1700 shown in FIG. 17. An additional loop is provided between the output of the op-amp 608 and the non-inverting input of the second op-amp 1706, comprising an adder 1802. The adders 1802 is configured to combine the third reference voltage VREF3 with the voltage at the output of the op-amp 608 and provide a compensated third reference voltage VREF3′ to the non-inverting input of the second op-amp 1706. The compensated third reference voltage VREF3′ is thus compensated so as to maintain the working electrode WE at the desired potential.

In embodiments described herein, the double layer capacitance CDL of the electrochemical cells 100, 300 are used as integrating elements in various integrators. By decoding an output of those integrators, for example by building an ADC around those integrators, a state or characteristic of the cells 100, 300 can be ascertained. It will be appreciated that embodiments of the disclosure are not limited to electrochemical cells (or other sensors) which are capacitive in nature. Any sensor having a reactive impedance may be provided in place of the exemplary cells 100, 300. For example, whilst in the examples described herein the electrochemical cells 100, 300 provide integrating capacitance in the feedforward loop of an integrator, in other embodiments sensors having inductive properties may provide an integrating inductance in a feedback loop of an integrator. Non-limiting examples of sensors having a reactive impedance include electrochemical cells (such as those described herein as well as gas sensors), inductive sensors, capacitive sensors, hall effect sensors, strain gauges, displacement sensors and level sensors.

The skilled person will recognise that some aspects of the above-described apparatus and methods may be embodied as processor control code, for example on a non-volatile carrier medium such as a disk, CD- or DVD-ROM, programmed memory such as read only memory (Firmware), or on a data carrier such as an optical or electrical signal carrier. For many applications embodiments of the invention will be implemented on a DSP (Digital Signal Processor), ASIC (Application Specific Integrated Circuit) or FPGA (Field Programmable Gate Array). Thus the code may comprise conventional program code or microcode or, for example code for setting up or controlling an ASIC or FPGA. The code may also comprise code for dynamically configuring re-configurable apparatus such as re-programmable logic gate arrays. Similarly the code may comprise code for a hardware description language such as Verilog TM or VHDL (Very high-speed integrated circuit Hardware Description Language). As the skilled person will appreciate, the code may be distributed between a plurality of coupled components in communication with one another. Where appropriate, the embodiments may also be implemented using code running on a field-(re) programmable analogue array or similar device in order to configure analogue hardware.

Note that as used herein the term module shall be used to refer to a functional unit or block which may be implemented at least partly by dedicated hardware components such as custom defined circuitry and/or at least partly be implemented by one or more software processors or appropriate code running on a suitable general-purpose processor or the like. A module may itself comprise other modules or functional units. A module may be provided by multiple components or sub-modules which need not be co-located and could be provided on different integrated circuits and/or running on different processors.

Embodiments may be implemented in a host device, especially a portable and/or battery powered host device. Non-limiting examples of such a host device include a mobile computing device for example a laptop or tablet computer, a games console, a remote control device, a home automation controller or a domestic appliance including a domestic temperature or lighting control system, a toy, a machine such as a robot, an audio player, a video player, or a mobile telephone for example a smartphone. Further non-limiting examples include wearable devices, such as medical wearable device (e.g., continuous analyte monitors (e.g., glucose monitors), smart watches, augmented reality headsets, virtual reality headsets and smart glasses. Further non-limiting examples include implantable medical devices, such as pacemakers and other neural stimulation devices.

It should be noted that the above-mentioned embodiments illustrate rather than limit the invention, and that those skilled in the art will be able to design many alternative embodiments without departing from the scope of the appended claims. The word “comprising” does not exclude the presence of elements or steps other than those listed in a claim, “a” or “an” does not exclude a plurality, and a single feature or other unit may fulfil the functions of several units recited in the claims. Any reference numerals or labels in the claims shall not be construed so as to limit their scope.

As used herein, when two or more elements are referred to as “coupled” to one another, such term indicates that such two or more elements are in electronic communication or mechanical communication, as applicable, whether connected indirectly or directly, with or without intervening elements.

This disclosure encompasses all changes, substitutions, variations, alterations, and modifications to the example embodiments herein that a person having ordinary skill in the art would comprehend. Similarly, where appropriate, the appended claims encompass all changes, substitutions, variations, alterations, and modifications to the example embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative. Accordingly, modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described above.

Unless otherwise specifically noted, articles depicted in the drawings are not necessarily drawn to scale.

All examples and conditional language recited herein are intended for pedagogical objects to aid the reader in understanding the disclosure and the concepts contributed by the inventor to furthering the art, and are construed as being without limitation to such specifically recited examples and conditions. Although embodiments of the present disclosure have been described in detail, it should be understood that various changes, substitutions, and alterations could be made hereto without departing from the spirit and scope of the disclosure.

Although specific advantages have been enumerated above, various embodiments may include some, none, or all of the enumerated advantages. Additionally, other technical advantages may become readily apparent to one of ordinary skill in the art after review of the foregoing figures and description.

To aid the Patent Office and any readers of any patent issued on this application in interpreting the claims appended hereto, applicants wish to note that they do not intend any of the appended claims or claim elements to invoke 35 U.S.C. § 112 (f) unless the words “means for” or “step for” are explicitly used in the particular claim.

1. Circuitry for measuring a characteristic of a sensor having a reactive impedance, the circuitry comprising:
a first integrator having a first integrator input, a first integrator output, and an integrating element coupled between the first integrator input and the first integrator output, the integrating element comprising the sensor; and
processing circuitry configured to:
determine a state of the sensor based on an output signal derived from the first integrator output.

2. The circuitry of claim 1, wherein the state of the sensor comprises a characteristic of the sensor.

3. The circuitry of claim 2, wherein the processing circuitry is configured to:
low-pass filter the output signal to determine a first aspect of the state of the sensor.

4. The circuitry of claim 1, wherein the processing circuitry is configured to:
high-pass filter the output signal to determine a second aspect of the state of the sensor.

5. The circuitry of claim 1, wherein the processing circuitry is configured to:
band-pass filter the output signal to determine a third aspect of the state of the sensor at one or more band-pass frequencies defined by the band-pass filtering.

6. The circuitry of claim 1, wherein the processing circuitry is configured to:
apply a Fourier transform to the output signal to determine the state of the sensor as a function of frequency.

7. The circuitry of claim 1, wherein, during derivation of the output signal, the processing circuitry is configured to hold first and second electrodes of the sensor at first and second predetermined voltages.

8. The circuitry of claim 1, wherein the sensor is a capacitive sensor or an inductive sensor.

9. (canceled)

10. The circuitry of claim 1, comprising:
an analog-to-digital converter (ADC), comprising:
an ADC output configured to output the output signal; and
the first integrator.

11. (canceled)

12. The circuitry of claim 10, comprising:
a digital-to-analog converter (DAC) provided in a feedback path between the ADC output and the first integrator input.

13. The circuitry of claim 1, wherein the processing circuitry is configured to:
derive a noise transfer function of the ADC from the output signal; and
determine the state of the sensor based on the noise transfer function.

14. The circuitry of claim 12, wherein the ADC comprises a quantizer comprising a quantiser input coupled to the first integrator output and a quantiser output coupled to the ADC output, wherein the quantiser is configured to derive the output signal based on the first integrator input.

15.-16. (canceled)

17. The circuitry of claim 14, further comprising control circuitry configured to adapt a hysteresis of the comparator based on a limit cycle frequency or limit cycle period of the first quantizer output.

18. The circuitry of claim 14, wherein the quantizer is a multi-bit quantiser and wherein the DAC comprises a finite impulse response DAC.

19. The circuitry of claim 10, further comprising dither circuitry configured to add noise at one or more of:
the first integrator input;
the ADC output;
the first integrator output; and
the feedback path between the ADC input and the ADC output.

20. The circuitry of claim 14, further comprising a second integrator having a second integrator input coupled to the first integrator output and a second integrator output coupled to the quantiser input.

21. The circuitry of claim 1, wherein the sensor comprises an electrochemical cell.

22. (canceled)

23. Circuitry of claim 21, wherein the electrochemical cell comprises a potentiostat comprising at least one working electrode and at least one counter electrode, wherein the at least one counter electrode is coupled to the first integrator input and the at least one working electrode is coupled to the first integrator output.

24. (canceled)

25. Circuitry of claim 21, wherein the electrochemical cell comprises a potentiostat comprising at least one working electrode, at least one counter electrode, and a reference electrode, wherein the reference electrode is coupled to the first integrator input and the at least one working electrode is coupled to the first integrator output.

26.-27. (canceled)

28. Circuitry of claim 21, wherein the electrochemical cell comprises a battery comprising an anode coupled to the first integrator input and a cathode coupled to the first integrator output.

29. Circuitry for measuring a characteristic of a sensor having a reactive impedance, the circuitry comprising:
a first integrator having a first integrator input, a first integrator output, and an integrating element coupled between the first integrator input and the first integrator output, the integrating element comprising the sensor; and
processing circuitry configured to decode an output signal derived from the first integrator output to determine a state of the sensor.

30. Circuitry comprising:
a sensor having a reactive impedance;
the sensor arranged as an integrating element of the circuitry;
wherein an integrated output of the integrating element is decoded to determine a state of the sensor.

31. An electronic device, comprising the circuitry of claim 1, wherein the device comprises one of a continuous glucose monitor, a wearable device, a medical device, an augmented reality headset, a virtual reality headset, a mobile computing device, a laptop computer, a tablet computer, a games console, a remote control device, a home automation controller, a domestic appliance, a toy, a robot, an audio player, a video player, a mobile telephone, and a smartphone.

32.-33. (canceled)
